(19) United States
(12) Patent Application Publication
(10) Pub. No.: US 2026/0092847 A1
(43) Pub. Date: Apr. 2, 2026

(54) DEVICE AND METHOD FOR ASSESSING REDUCTION DEGREE OF IRON ORE IN A MIXED REDUCING ATMOSPHERE

(71) Applicant: NATIONAL CHENG KUNG UNIVERSITY, TAINAN CITY (TW)

(72) Inventors: Shih-kang LIN, TAINAN CITY (TW); Yu-ning CHIU, TAINAN CITY (TW); Tsung-yen HUANG, KAOHSIUNG (TW); Jia-shyan SHIAU, KAOHSIUNG (TW); Kai-chun CHANG, TAINAN CITY (TW); Shan-wen DU, KAOHSIUNG (TW)

(73) Assignee: NATIONAL CHENG KUNG UNIVERSITY, TAINAN CITY (TW)

(21) Appl. No.: 19/267,558

(22) Filed: Jul. 13, 2025

(30) Foreign Application Priority Data
Sep. 30, 2024 (TW) 113137317

(51) Int. Cl. G01N 5/04; G01N 17/00; G01N 33/00; G01N 33/24

(52) U.S. Cl. CPC G01N 5/04; G01N 17/006; G01N 33/0013; G01N 33/24

(57) ABSTRACT

A device and a method for assessing reduction degree of iron ore in a mixed reducing atmosphere are provided. The device comprises: a hermetic heating device for heating a reaction sample; an air intake device configured to introduce an inert gas and/or a reducing gas into the hermetic heating device; a gas analysis device configured to analyze a part of exhaust gas; a thermogravimetric analysis device configured to analyze a weight change caused by reduction; a gas convection buffer section configured to mitigate disturbance of gas reflux on measurement of the thermogravimetric analysis device; and a gas split device configured to split the exhaust gas thereby maintaining the exhaust gas at a constant flow rate or a constant flow. The reduction degree of the iron ore in different reducing atmospheres is assessed according to a measured value of the gas analysis device and the thermogravimetric analysis device.

### CROSS-REFERENCE TO RELATED APPLICATIONS

This application claims the benefit of Taiwan Patent Application No 113137317, filed on Sep. 30, 2024, which is hereby incorporated by reference in its entirety herein.

### FIELD OF INVENTION

The present disclosure relates to a device and method for assessing reduction degree of iron ore, and in particular to a device and method for assessing reduction degree of iron ore in a mixed reducing atmosphere.

### BACKGROUND OF INVENTION

In conventional steelmaking processes, carbon-based metallurgical methods result in the emission of large amounts of carbon dioxide, which is a major contributor to global warming. With anticipated implementation of carbon tax systems in international markets, the continued development of carbon-based metallurgical methods is expected to be increasingly disadvantageous.

Alternative technical solutions generally advocate use of hydrogen as a substitute reducing agent, as the by-product of hydrogen-based reduction is harmless water (H₂O), whose applicability and environmental benefits have been extensively validated. Replacing carbon with hydrogen in the steelmaking process could fundamentally eliminate environmental issues caused by carbon dioxide emissions.

In the prior art, several hydrogen-based direct reduction iron (DRI) technologies have been developed in steel industries to reduce carbon dioxide emissions. Representative examples include HYL and Midrex processes, which utilize hydrogen-rich gas mixtures as reducing agents. Additionally, full-oxygen blast furnaces, top gas recycling systems, and COREX process are also characterized by the use of reducing gases with high concentrations of carbon monoxide and hydrogen. A mixed reducing atmosphere comprising a high concentration of hydrogen along with a certain proportion of carbon-based gases is used in these processes to perform reduction reactions. Therefore, evaluation of reduction rates of iron ore in hydrogen-carbon monoxide mixed gas atmospheres has become an important research topic. Although the reduction of iron oxides using pure hydrogen and pure carbon monoxide has been extensively studied in the past, reduction kinetics of hydrogen-carbon monoxide mixed gases under mixed conditions have not yet been sufficiently investigated.

During the reduction of iron ore using reducing gases, the reaction between iron oxide (FeO) and carbon monoxide (CO) can be represented by the following formula (1):

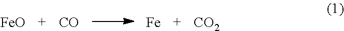

When a small amount of hydrogen is introduced as a reducing gas, the reduction reaction can proceed not only by formula (1), but also by the reaction with hydrogen as shown in the following formula (2):

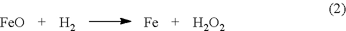

However, regardless of whether the reduction proceeds via formula (1) or formula (2), oxygen weight loss measured by conventional thermogravimetric analysis (TGA) techniques shows no difference. As a result, a specific reduction reaction pathway through which the iron ore is reduced cannot be identified. Furthermore, introduction of a small amount of hydrogen may also trigger a partial reverse water-gas shift reaction, represented by the following formula (3):

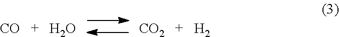

As a result, a combined reaction of formula (2) and formula (3) yields the same overall reaction as formula (1), which indicates that the reduction by hydrogen may proceed through a combination of the hydrogen reduction reaction (formula (2)) and the reverse water-gas shift reaction (formula (3)), rather than through formula (1) alone. Substantial challenges are introduced to the accurate evaluation of reduction of iron ore in mixed gas conditions as a result of differing reaction pathways with the same overall reaction formula. Consequently, developments of technical methods capable of precisely identifying the actual reaction pathways have become a critical factor in advancing hydrogen-based mixed gas reduction technologies. Methods or devices that enable independent quantification of reaction intensities induced by various reducing gases and their respective contributions to the degree of reduction are expected to possess substantial practical significance and applicability.

Conventional thermogravimetric analysis (TGA) techniques primarily rely on monitoring weight change of the reactants to track degree of reduction. While a reduction rate can be analyzed by the aforementioned techniques, an exact reduction reaction occurring cannot be specifically determined. In cases where multiple reducing gases are involved simultaneously, such as in a reduction using a hydrogen-carbon monoxide mixed gas, only an overall degree of reduction resulting from all reactions can be measured by the existing methods, without distinguishing the specific contributions of each individual reaction.

Limitations of conventional thermogravimetric analysis techniques significantly impede future developments of technologies involving hydrogen-carbon-based mixed gas reduction reactions. However, in the absence of the ability to differentiate the individual contributions of each reaction, a comprehensive understanding and precise control of the reduction process are substantially limited, resulting in difficulties in accurately controlling chemical reaction parameters to enhance production efficiency. In current hydrogen-based reduction technologies, hydrogen-carbon monoxide mixed gases are predominantly utilized. Therefore, accurate assessment of respective impacts of hydrogen and carbon monoxide on the reduction degree is essential for precise optimization of a gas composition ratio in mixed reducing gases, as well as for optimizing reaction conditions and process design.

Accordingly, it is necessary to provide a device and method for assessing reduction degree of iron ore in a mixed reducing atmosphere, so as to overcome problems present in conventional techniques.

### SUMMARY OF INVENTION

### Technical Problems

In view of the foregoing, a main purpose of the present disclosure is to provide a device and method for assessing reduction degree of iron ore in a mixed reducing atmosphere. The present disclosure aims to address difficulties in evaluating iron ore reduction reactions in mixed atmosphere by providing an innovative analytical device and method capable of accurately distinguishing and quantifying reaction contributions of multiple reducing gases, such as carbon monoxide and hydrogen, overcoming limitations of conventional thermogravimetric analysis.

Another purpose of the present disclosure is to provide a device and method for assessing reduction degree of iron ore in a mixed gas reducing environment. Differences in compositions of gases entering and exiting a furnace chamber can be accurately monitored and analyzed by combining novel thermogravimetric analysis and exhaust gas analysis according to the present disclosure, thereby enabling a direct assessment of the reduction degree of the iron ore. Real-time tracking and identification of compositional changes of multiple reducing gases, as well as instant assessment of reduction contributions from respective reactions, are enabled by the method of the present disclosure. Accordingly, even under complex mixed gas conditions, individual reduction rates (such as separately assessing contributions of CO—CO₂ and H₂-H₂O reduction reactions) can be precisely determined, thereby facilitating improved monitoring and control of reduction kinetics of mixed reducing gas.

### Technical Solutions

To achieve the above-mentioned purpose, the present disclosure provides a device for assessing reduction degree of iron ore in a mixed reducing atmosphere, comprising: a hermetic heating device, configured to heat a reaction sample; an air intake device, connected to the hermetic heating device, and configured to introduce an inert gas and/or a reducing gas into the hermetic heating device; a gas analysis device, configured to analyze a part of exhaust gas produced by the hermetic heating device; a thermogravimetric analysis device, configured to analyze a weight change of the hermetic heating device caused by reduction; a gas convection buffer section, disposed between the hermetic heating device and the thermogravimetric analysis device, and configured to mitigate disturbance caused by gas reflux on measurement of the thermogravimetric analysis device; and a gas split device, disposed between the hermetic heating device and the gas analysis device, and configured to split the exhaust gas produced by the hermetic heating device, thereby maintaining the part of exhaust gas introduced into the gas analysis device at a constant flow rate or a constant flow, wherein a reduction degree of iron ore of the reaction sample in different reducing atmosphere is respectively assessed according to a measured value of the gas analysis device and a measured value of the thermogravimetric analysis device.

In some embodiments of the present disclosure, the gas convection buffer section includes a gas convection buffer area and a constant pressure area, the constant pressure area is disposed near one side of the thermogravimetric analysis device, and the gas convection buffer area has a convection gas exhaust port configured to exhaust convection gas in the gas convection buffer area.

In some embodiments of the present disclosure, a pressure in the constant pressure area is controlled to be 1 atmosphere.

In some embodiments of the present disclosure, the hermetic heating device includes a reaction sample loading device configured to load the reaction sample, and the reaction sample is iron ore to be tested.

In some embodiments of the present disclosure, the constant pressure area includes a hermetic component, a connector is disposed inside the hermetic component, one end of the connector is connected to the thermogravimetric analysis device, and the other end of the connector is connected to the reaction sample loading device.

In addition, the present disclosure further provides a method for assessing reduction degree of iron ore in a mixed reducing atmosphere by using the device as mentioned above, comprising the following steps: providing a reaction sample in a hermetic heating device to heat the reaction sample; introducing an inert gas and/or a reducing gas into the hermetic heating device; and introducing a part of exhaust gas produced by the hermetic heating device into a gas analysis device, and assessing reduction degree of iron ore of the reaction sample in different reducing atmosphere respectively according to a measured value of the gas analysis device and a measured value of the thermogravimetric analysis device.

In some embodiments of the present disclosure, the reduction degree is obtained by assessing the reduction degree of the iron ore in different reducing atmosphere respectively by analyzing change in thermogravimetric weight loss to calculate the reduction degree of the iron ore using the thermogravimetric analysis device and by analyzing change in a composition of the exhaust gas using the gas analysis device.

In some embodiments of the present disclosure, assessing the reduction degree of the iron ore by the change in thermogravimetric weight loss is performed by the following formula:

$$Reduction Degree ( RD ) = [ \frac{0.111 \times {W}_{1}}{0.43 \times {W}_{2}} + \frac{{M}_{1} - {M}_{t}}{{M}_{0} \times ( 0.43 \times {W}_{2} )} ] \times 100 \% ,$$

wherein RD % represents the reduction degree of the iron ore (%); M₀ represents a total mass of the iron ore; W₁ represents FeO content of the iron ore (%); W₂ represents a total iron fraction (TFe) content of the iron ore (%); M₁ represents a total weight of the iron ore at the time t=0 of an experiment; and Mt represents a total weight of the iron ore at the t^{th} second of an experiment.

In some embodiments of the present disclosure, assessing the reduction degree of the iron ore in different reducing atmospheres respectively by utilizing gas change by analyzing the composition of the exhaust gas using the gas analysis device is performed by the following formula:

$$D \% ( CO ) = \frac{{N}_{C O} + 2 {N}_{CO 2} - {N}_{introduced CO} - 2 {N}_{introduced CO 2}}{{M}_{0} \times [ ( 0.430 \times {W}_{2} ) - ( 0.111 \times {W}_{1} ) ] / 16} \times 100 \% ,$$

$$RD \% ( H 2 ) = \frac{{N}_{H 2} + 2 {N}_{H 2 O} - {N}_{introduced H 2} - 2 {N}_{introduced H 2 O}}{{M}_{0} \times [ ( 0.430 \times {W}_{2} ) - ( 0.111 \times {W}_{1} ) ] / 16} \times 100 \% ,$$

wherein RD % represents the reduction degree of the iron ore (%); M₀ represents a total mass of the iron ore; N_{CO} represents a molar amount of CO gas contained in the exhaust gas; N_{CO2} represents a molar amount of CO₂ gas contained in the exhaust gas; N_{introduced CO} represents a molar amount of CO gas contained in the introduced gas; N_{introduced CO2} represents a molar amount of CO₂ gas contained in the introduced gas; NH₂ represents a molar amount of H₂ gas contained in the exhaust gas; N_{H2O} represents a molar amount of H₂O gas contained in the exhaust gas; N_{introduced H2} represents a molar amount of H₂ gas contained in the introduced gas; N_{introduced H2O} represents a molar amount of H₂O gas contained in the introduced gas; W₁ represents FeO content of the iron ore (%); and W₂ represents a total iron content (TFe) of the iron ore (%).

In some embodiments of the present disclosure, an accuracy of the assessing of the reduction degree is verified by comparing the measured value of the gas analysis device with the measured value of the thermogravimetric analysis device.

### Beneficial Effects

In the present disclosure, the assessment of the reduction degree is not limited by the completion status of the reduction of the iron ore, thereby providing greater flexibility and adaptability in the application of the technique. Accurate assessment of the reduction process can also be achieved even before full reduction of the iron ore is completed or while the reaction is still ongoing, thereby allowing specific process parameters to be adjusted and production efficiency to be enhanced.

### DESCRIPTION OF DRAWINGS

In order to more clearly illustrate the above contents of the present disclosure, the following is a detailed description of the preferred embodiments with reference to the accompanying drawings:

FIG. 1 is a schematic diagram of a device for assessing reduction degree of iron ore in a mixed reducing atmosphere according to an embodiment of the present disclosure.

FIG. 2 is a schematic diagram of a device for assessing reduction degree of iron ore in a mixed reducing atmosphere according to an specific embodiment of the present disclosure.

FIG. 3 is a perspective view of FIG. 1 including a hermetic heating device, a gas convection buffer section, and a thermogravimetric analysis device.

FIG. 4 is a cross-sectional view of FIG. 3 including a hermetic heating device, a gas convection buffer section, and a thermogravimetric analysis device.

FIG. 5 is a perspective view of another embodiment of the present disclosure including a gas convection buffer section and a thermogravimetric analysis device.

FIG. 6 is a flow diagram illustrating a method for assessing reduction degree of iron ore in a mixed reducing atmosphere according to an embodiment of the present disclosure.

FIG. 7 shows reduction degrees of the iron ore under four different isothermal conditions in a mixed reducing atmosphere (35% CO, 10% H₂, 55% Ar), respectively determined by thermogravimetric analysis and mass spectrometry according to an embodiment of the present disclosure.

FIG. 8 illustrates respective contributions of CO and H₂ to the reduction degrees of the iron ore, assessed based on exhaust gas analysis data in a mixed reducing atmosphere (35% CO, 10% H₂, 55% Ar) according to an embodiment of the present disclosure.

FIG. 9 is a schematic diagram showing effects of thermal disturbance on thermogravimetric analysis in the prior art without disturbance elimination and in the present disclosure with disturbance elimination.

### DETAILED DESCRIPTION OF PREFERRED EMBODIMENTS

In order to describe the technical solutions of the present disclosure more clearly, numerous specific details are provided in the following specific embodiments. Apparently, the present disclosure can be practiced without certain specific details.

The core of the present disclosure lies in the combination of thermogravimetric analysis technologies and exhaust gas analysis monitoring equipment, by which the composition, concentration, and flow rate of the gas can be accurately measured. The key information regarding the reduction degree of the iron ore can be accurately derived by analyzing the composition of the exhaust gas. The method of the present disclosure is particularly suitable for reduction reactions under mixed atmospheres. Technical barriers encountered in existing solutions can be effectively overcome, and the following advantages can be achieved:

### (1) Improvement of Accuracy of Mixed-Gas Reduction Analysis:

Accurate differentiation of the contribution of each reduction reaction is enabled, and the reduction degree can be assessed by means of thermogravimetric analysis and exhaust gas analysis techniques for comparative purposes. The reliability of the analysis is enhanced, and deeper insights into the reaction mechanisms can be obtained by cross-verification.

(2) Elimination of the Interference Caused by Weight Changes from Non-Reduction Reactions:

In addition to reduction reactions, non-reduction reactions such as the gasification of coke (CO₂+C→2CO) and the carburization of metallic iron forming Fe₃C may also result in weight changes of the reactants. These interfering factors, which have been difficult to eliminate in past experiments, can be thoroughly addressed by the device and method of the present disclosure, thereby ensuring purity and accuracy of measurement results.

### (3) Elimination of Disturbances Caused by the Pendulum Motion of the Reaction Sample Loading Device Due to Thermal Convection:

In conventional methods, the reaction sample loading device is typically suspended within the furnace chamber by a thin wire. The configuration is susceptible to airflow-induced convection, causing slight oscillations of the device. The measurement accuracy of thermogravimetric analysis may be indirectly influenced and errors may be introduced by such oscillations. These problems as mentioned above are eliminated by the present disclosure, whereby measurement errors resulting from oscillation of the reaction sample loading device are completely overcome, and the accuracy and reliability of experimental data are significantly improved.

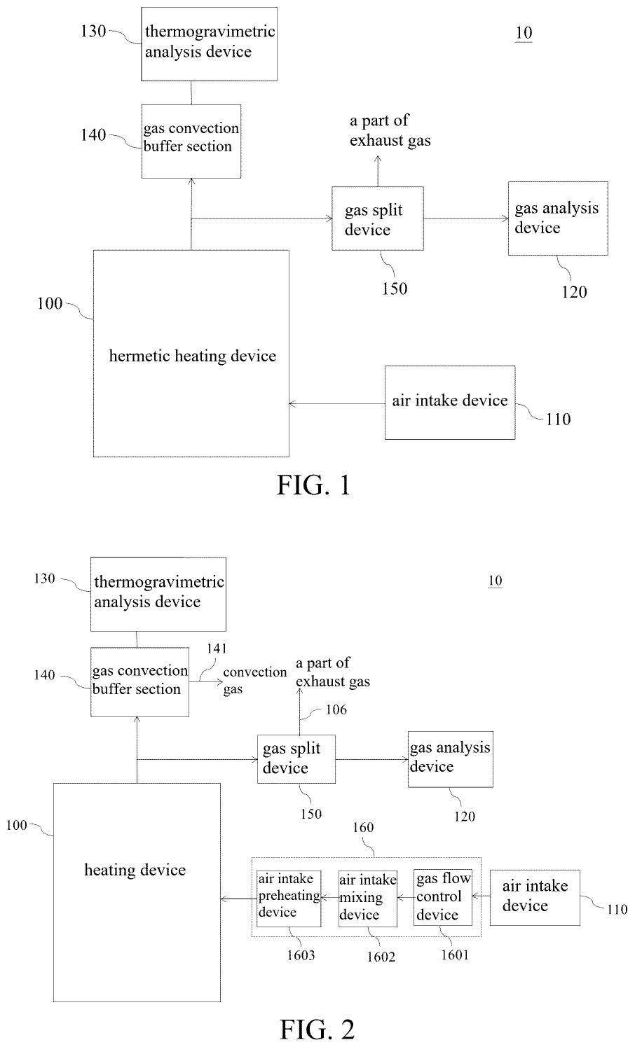

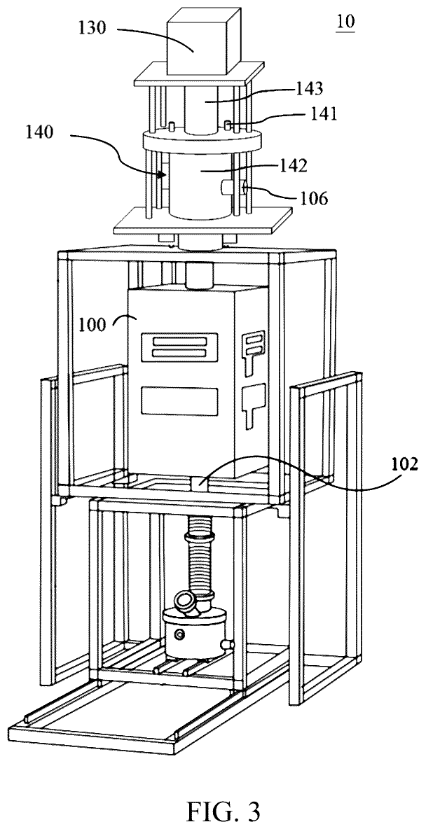

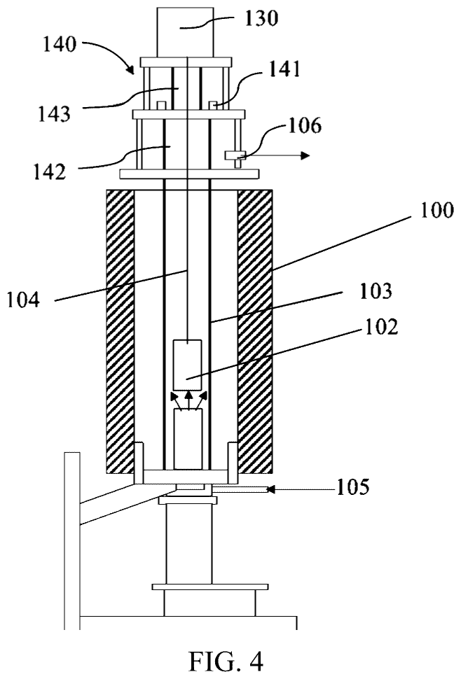

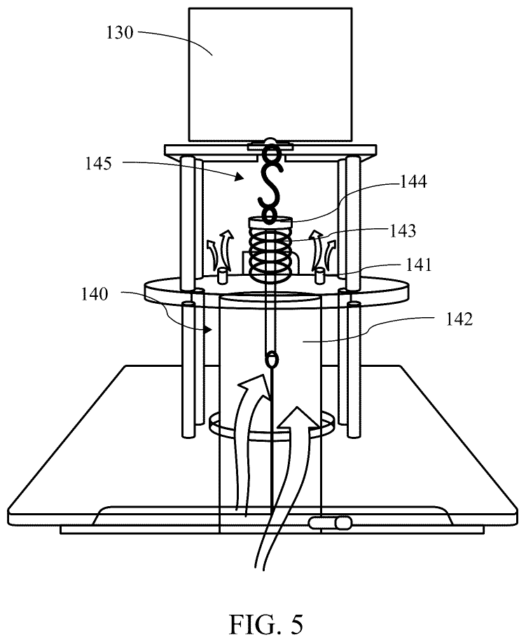

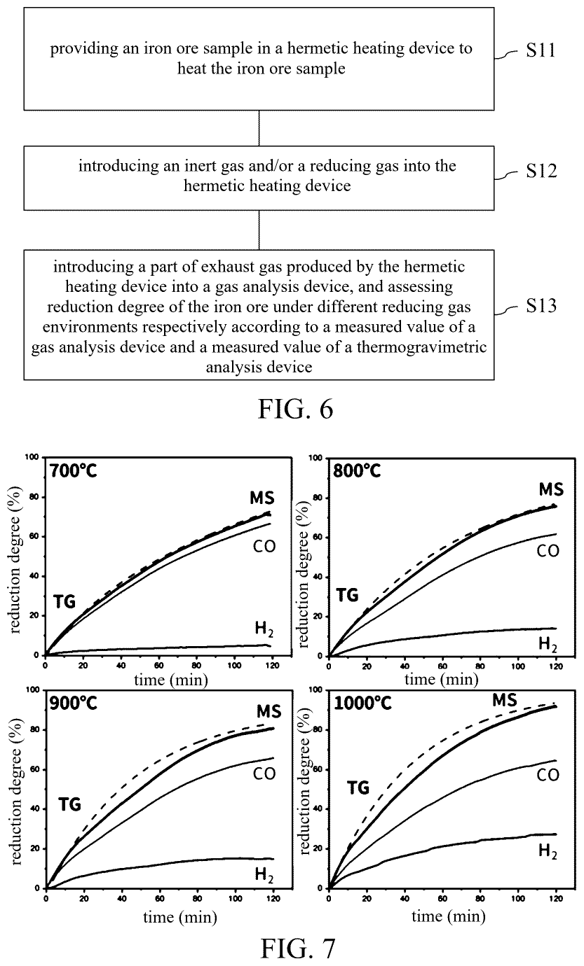

Refer to FIG. 1 to FIG. 5. FIG. 1 is a schematic diagram of a device assessing reduction degree of iron ore in a mixed reducing atmosphere according to an embodiment of the present disclosure. FIG. 2 is a schematic diagram of a device for assessing reduction degree of iron ore in a mixed reducing atmosphere according to a specific embodiment of the present disclosure. FIG. 3 is a perspective view of FIG. 1 including a hermetic heating device, a gas convection buffer section, and a thermogravimetric analysis device. FIG. 4 is a cross-sectional view of FIG. 3 including a hermetic heating device, a gas convection buffer section, and a thermogravimetric analysis device. FIG. 5 is a perspective view of another embodiment of the present disclosure including a gas convection buffer section and a thermogravimetric analysis device. FIG. 6 is a flow diagram illustrating a method for assessing reduction degree of iron ore in a mixed reducing atmosphere according to an embodiment of the present disclosure.

As shown in FIG. 1, a device 10 for assessing reduction degree of iron ore in a mixed reducing atmosphere of the present disclosure comprises: an hermetic heating device 100 configured to heat a reaction sample; an air intake device 110 connected to the hermetic heating device 100, and configured to introduce an inert gas and/or a reducing gas into the hermetic heating device 100; a gas analysis device 120 configured to analyze a part of exhaust gas produced by the hermetic heating device 100; a thermogravimetric analysis device 130 configured to analyze a weight change of the hermetic heating device 100 caused by reduction; a gas convection buffer section 140, disposed between the hermetic heating device 100 and the thermogravimetric analysis device 130, and configured to mitigate disturbance caused by gas reflux on measurement of the thermogravimetric analysis device 130; a gas split device 150, disposed between the hermetic heating device 100 and the gas analysis device 120, and configured to split the exhaust gas produced by the hermetic heating device 100, thereby maintaining the part of exhaust gas introduced into the gas analysis device 120 at a constant flow rate or a constant flow, wherein a reduction degree of iron ore of the reaction sample in different reducing atmospheres is respectively assessed according to a measured value of the gas analysis device 120 and a measured value of the thermogravimetric analysis device 130.

Specifically, as shown in FIG. 2, the air intake device 110 may be, but is not limited to, at least one gas cylinder or gas storage tank, or other equipment that can be used to introduce at least one inert gas and/or reducing gas into the hermetic heating device 100. The air intake device 110 is optionally connected to an air intake pre-treatment device 160 (for example, but not limited to, a gas flow control device 1601, an air intake mixing device 1602, an air intake preheating device 1603). Flow of the air intake device 110 can be controlled by the gas flow control device 1601. Multiple gas sources can be mixed by the air intake mixing device 1602. The inert gas and/or reducing gas introduced into the hermetic heating device 100 can be preheated by the air intake preheating device 1603.

In addition, the gas split device 150 may comprise an exhaust gas inlet flow rate/pressure control device, which ensures that a part of the exhaust gas entering the gas analysis device 120 is maintained at a constant flow rate or a constant flow, while the remaining exhaust gas is discharged through an exhaust emission branch 106. Convection gas within the gas convection buffer section 140 may be discharged through a convection gas outlet 141.

In addition, as shown in FIG. 3 and FIG. 4, the hermetic heating device 100 may comprise a reaction sample loading device 102 and a hermetic reaction chamber 103. In one example, the reaction sample loading device 102 may include a crucible, and the hermetic reaction chamber 103 may be configured as a tube adapted to maintain a sealed reaction environment. The hermetic heating device 100 is configured to provide the high temperature conditions necessary for a chemical reaction, and to maintain both an elevated temperature and the required sealed system via the hermetic reaction chamber 103.

The reaction sample loading device 102 may be loaded with iron ore of a fixed weight and a specific particle size for reaction. The thermogravimetric analysis device 130 (for example, but not limited to, a weight sensor) is configured to track the weight change of the reaction sample loading device 102 during the reaction. The thermogravimetric analysis device 130 may be connected to the reaction sample loading device 102 via a connection member 104 (for example, but not limited to, a hanging wire), thereby achieving real-time measurement of the weight change of the reaction sample loading device 102. During the test, the gas required for the reaction may be provided by the air intake device 110. After moisture pretreatment, the intake gas can be passed through the intake connecting pipeline and the composition and flow of the intake gas can be adjusted in the gas flow control device 1601. The specified gas composition is mixed by an air intake mixing device 1602. The mixed gas is preheated by an air intake preheating device 1603 before entering the hermetic heating device through an air intake port 105. To enable real-time monitoring of the reaction conditions within the furnace chamber, an equipment may be equipped with an exhaust gas treatment system. The post-reaction gases are discharged from an outlet, then split and collected via a manifold pipeline. A portion of the exhaust gas is directed through an exhaust emission branch 106 for waste gas treatment, while a smaller portion is routed through a gas split device 150 into a gas analysis device 120 for exhaust gas analysis. The gas flow rate and temperature are stabilized by the gas split device 150 to prevent condensation of water vapor within pipelines. Ultimately, the gas analysis device 120 is configured to analyze compositions of the gas. All measured data, including exhaust gas analysis and weight change data, may be recorded and subsequently processed by a computer. By a highly integrated design of the equipment, evaluation of the reduction degree of the iron ore can be achieved under various temperatures and reducing atmosphere conditions.

In addition, a highly hermetic reaction chamber (i.e., the hermetic heating device 100) is specially designed in the present disclosure. A gas convection buffer section 140 is provided above the exhaust gas outlet, so as to effectively mitigate disturbances caused by high-pressure, high-velocity, and high-temperature gas backflow on the reaction sample loading device 102, while deceleration, pressure reduction, and cooling effects can be simultaneously achieved. After pressure reduction, the gas is safely discharged through the convection gas outlet 141. The gas convection buffer section 140 may include a gas convection buffer area 142 and a constant pressure area 143. The gas convection buffer area 142 may be a columnar tubular structure containing an internal gas convection buffer chamber. The constant pressure area 143 is disposed above the gas convection buffer area 142. The constant pressure area 143 may be formed by a section of hermetic high-temperature hose, which is connected to the columnar tubular structure of the gas convection buffer area 142. The gas in the constant pressure area 143 may, for example, be maintained at approximately room temperature and one atmosphere of pressure.

Refer to FIG. 5. In one embodiment, an upper side of the constant pressure area 143 may be sealed with a hermetic upper cover 144, which is connected via a connecting component 145 (e.g., an S-shaped hook and a suspension rod), and the thermogravimetric analysis device 130 is connected to the reaction sample loading device 102 by a wire, so as to prevent interference from airflow-induced disturbances. The reaction sample loading device 102 is suspended by the suspension rod in the hermetic heating device 100, thereby enabling the thermogravimetric analysis device 130 to accurately detect weight variations throughout the reaction. In addition, a protective cover can be provided outside the thermogravimetric analysis device 130 to avoid the influence of atmospheric airflow on the thermogravimetric analysis device 130. The reaction gas convection route shown in FIG. 5 illustrates a path of the gas from the hermetic reaction chamber 103 in the hermetic heating device 100 to the gas convection buffer section 140, and finally discharged from the convection gas outlet 141 of the gas convection buffer area 142.

Entry of external gases and leakage of reaction gases can be effectively prevented by the highly hermetic reaction chamber in the hermetic heating device 100 of the present disclosure, thereby ensuring accuracy of analysis the exhaust gas. However, the inventors have found that, compared to conventional non-hermetic chamber designs, the configuration is more prone to interference with the weight sensor of the upper thermogravimetric analysis device 130 caused by gas convection, resulting in measurement errors in the thermogravimetric analysis. Therefore, the gas convection buffer area 142 is particularly provided above the exhaust emission branch 106 in the present disclosure to effectively reduce disturbances caused by the high-pressure, high-flow-rate, and high-temperature gas backflow to the reaction sample loading device 102. The effects of deceleration, pressure reduction, and temperature reduction are thereby achieved, and thermal damage to the electronic components of the weight sensor caused by the high-temperature gas can be prevented. The depressurized gas is discharged through the convection gas outlet 141, which is connected to the constant pressure area 143, such as a hermetic high-temperature hose. At the stage, the gas within the constant pressure area 143 is mostly close to the room temperature and atmospheric pressure, significantly reducing airflow interference with the weight sensor to a negligible level. The reaction sample loading device 102 inside the hermetic chamber is connected to the thermogravimetric analysis device 130 via a connecting component, thereby preventing any influence from airflow disturbances.

### Reactant

The primary reactant is iron ore, which may comprise hematite (Fe₂O₃), magnetite (Fe₃O₄), and/or wüstite (FeO), and may further contain oxides of silicon, aluminum, magnesium, and calcium. In order to ensure uniform particle size of the reactant and to eliminate variations in reaction rate measurements caused by particle size non-uniformity, the iron ore reactant may be screened using sieves of 7.0 mm, 9.5 mm, 12.7 mm, and 15.9 mm mesh sizes, so as to obtain four categories of particle sizes. The specific particle size ranges may include: 7.0-9.5 mm, 9.5-12.7 mm, 12.7-15.9 mm, and greater than 15.9 mm. The iron ore reactant may be spread in a single layer, without overlapping, and placed within a reaction sample loading device 102 (e.g., a crucible) having an inner diameter of 55 mm. Such an arrangement may prevent gas flow obstruction caused by stacking, thereby ensuring accurate measurement of the reaction rate. Additionally, a bottom of the reaction sample loading device 102 may be specifically designed with strip-shaped grid openings to allow uniform introduction of reaction gas from the bottom of the reaction sample loading device 102, thereby promoting sufficient contact between the reaction gas and the reactant for an chemical reaction.

The present disclosure further provides a method for assessing reduction degree of iron ore in a mixed reducing atmosphere by using the device as described above, comprising the following steps: (S11) providing a reaction sample in the hermetic heating device to heat the reaction sample; (S12) introducing an inert gas and/or a reducing gas into the hermetic heating device; and (S13) introducing a part of exhaust gas produced by the hermetic heating device into the gas analysis device, and assessing the reduction degree of the iron ore of the reaction sample in the different reducing atmospheres respectively according to a measured value of the gas analysis device and a measured value of the thermogravimetric analysis device.

Step S11 may further comprises the following process. The screened iron ore reactant (in this example, the iron ore with a particle size ranging from 9.5 to 12.7 mm and a weight of 34.0 g) is placed into the reaction sample loading device 102 (e.g., an alumina crucible), and the described placing method is followed to eliminate interference caused by variations in particle size on the reduction rate measurement. Subsequently, the reaction sample loading device 102 loaded with the reactant is placed into the hermetic heating device 100. The gas preheating and pretreatment equipment is initiated, and the flow rate, pressure, and temperature of the exhaust gas discharge are set. The gas analysis device is calibrated to ensure that the exhaust gas composition measured at the furnace chamber outlet of the hermetic heating device 100 is consistent with the input atmosphere composition before experiment begins. Additionally, a weight sensor of the thermogravimetric analysis device 130 is reset to zero to guarantee accurate reflection of mass changes during the reduction process of the iron ore.

Step S12 may further comprises the following process. The air intake device 110 is controlled to continuously supply nitrogen or argon inert gas into the hermetic heating device 100 at a heating rate of 10° C./min. When a predetermined experimental temperature is reached, the atmosphere is switched to a specific reducing environment and maintained for 120 minutes. When the experimental temperature attains a set isothermal condition (in the example, four temperature levels being applied sequentially: 700° C., 800° C., 900° C., and 1000° C.), the gas is switched to a predetermined mixed reducing atmosphere composed of 35% carbon monoxide, 10% hydrogen, and 55% argon, thereby simulating a blast furnace ironmaking environment. A flow rate is set to 5 liters per minute, and a temperature is held for 120 minutes. Afterwards, argon is introduced at 5 liters per minute for cooling.

Step S11 may further comprise the following processes. Data acquired and recorded by a computer in real time are assessed. The thermogravimetric data from the thermogravimetric analysis device 130 and the exhaust gas analysis data from the gas analysis device (e.g., mass spectrometer (MS) data) are converted into the reduction degree, and the reduction degree curve of the iron ore is generated. Upon completion of the experiment, material is removed from the reaction sample loading device 102 and subjected to a secondary weight measurement to verify consistency with the data recorded by the thermogravimetric analysis device 130, with an error controlled within 0.2 grams. Subsequently, material characterization techniques such as X-ray diffraction analysis (XRD) are performed to confirm that the phase fractions of the iron ore products correspond to the measured reduction degree data.

### Reduction Product Results

### Removal of Reduction Products and Data Processing

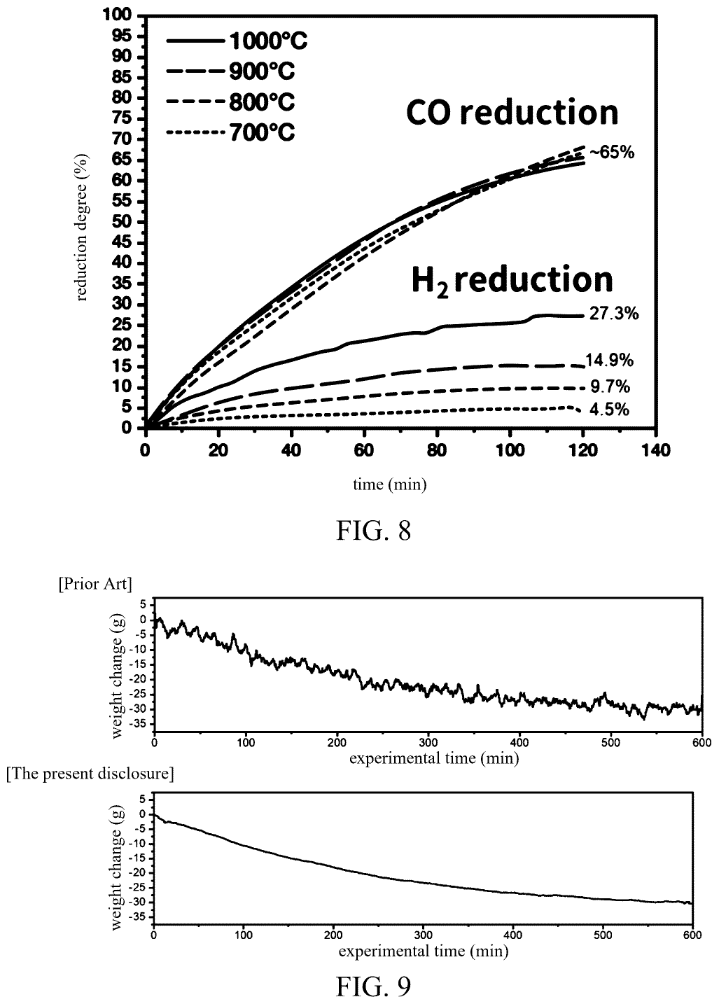

The reduction reaction according to the present disclosure is performed to acquire thermogravimetric change data and exhaust gas composition data for analysis, enabling an assessment of the reduction degree variation of the iron ore over time. The results are illustrated in FIG. 7 and FIG. 8.

The reduction degree (RD %) of the iron ore is calculated based on thermogravimetric weight loss changes:

$$Reduction Degree ( RD ) = [ \frac{0.111 \times {W}_{1}}{0.43 \times {W}_{2}} + \frac{{M}_{1} - {M}_{t}}{{M}_{0} \times ( 0.43 \times {W}_{2} )} ] \times 100 \% ,$$

RD % represents the reduction degree of the iron ore (%); M₀ represents a total mass of the iron ore; W₁ represents FeO content in iron ore (%); W₂ represents a total iron fraction (TFe) content of the iron ore (%); M₁ represents a total weight of the iron ore at the time t=0 of an experiment; and Mt represents a total weight of iron ore at the t^{th} second of an experiment.

The atmosphere change is used to assess the reduction degree (RD %) of the iron ore caused by different reducing atmospheres:

$$RD \% ( CO ) = \frac{{N}_{C O} + 2 {N}_{CO 2} - {N}_{introduced CO} - 2 {N}_{introduced CO 2}}{{M}_{0} \times [ ( 0.430 \times {W}_{2} ) - ( 0.111 \times {W}_{1} ) ] / 16} \times 100 \%$$

$$RD \% ( H 2 ) = \frac{{N}_{H 2} + 2 {N}_{H 2 O} - {N}_{introduced H 2} - 2 {N}_{introduced H 2 O}}{{M}_{0} \times [ ( 0.430 \times {W}_{2} ) - ( 0.111 \times {W}_{1} ) ] / 16} \times 100 \%$$

RD % represents the reduction degree of the iron ore (%); M₀ represents a total mass of the iron ore; N_{CO} represents a molar amount of CO gas contained in the exhaust gas; N_{CO2} represents a molar amount of C_{O2} gas contained in the exhaust gas; N_{introduced CO} represents a molar amount of CO gas contained in the introduced gas; N_{introduced CO2} represents a molar amount of CO₂ gas contained in the introduced gas; NH₂ represents a molar amount of H₂ gas contained in the exhaust gas; N_{H2O} represents a molar amount of H₂O gas contained in the exhaust gas; N_{introduced H2} represents a molar amount of H₂ gas contained in the introduced gas; N_{introduced H2O} represents a molar amount of H₂O gas contained in the introduced gas; W₁ represents FeO content of the iron ore (%); and W₂ represents a total iron content (TFe) of the iron ore (%).

As shown in FIG. 7 and FIG. 8, a comparison is made between a reduction degree determined based on a weight change measured using a thermogravimetric analysis device (TGA), represented by a black dashed line, and a reduction degree determined based on an exhaust gas change measured using a mass spectrometer (MS), represented by a dark solid line. If a deviation between these two measurement methods is less than 5%, the data obtained from the reduction experiment may be regarded as reliable. Since thermogravimetric analysis data are easily affected by weight fluctuations and thermal disturbances arising from non-reduction reactions, the final reduction degree primarily relies on exhaust gas analysis data. By employing these two distinct assessment methods, accuracy of the reduction degree determination can be cross-validated. In the present disclosure, individual contributions to the reduction degree caused by different gas atmosphere compositions can be independently determined by use of the reduction degree assessed using exhaust gas analysis, which provides a significant advantage for evaluation of reduction reactions of the iron ore under mixed atmospheres in the future, allowing clear identification of the respective contributions to the reduction degree from the carbon monoxide reduction and hydrogen reduction in a carbon monoxide-hydrogen mixed atmosphere.

FIG. 9 illustrates the influence of thermal disturbance on thermogravimetric analysis before and after mitigation. As shown in FIG. 9, in the present disclosure, an impact of airflow convection and crucible oscillation (pendulum effect) induced by furnace heating on measurements by a weight sensor of the thermogravimetric analysis device is effectively eliminated.

### Advantages of the Present Disclosure

### 1. Combination of Thermogravimetric and Exhaust Gas Analysis:

In the present disclosure, thermogravimetric analysis (TGA) equipment is combined with exhaust gas analysis equipment, thereby enabling simultaneous physical and chemical analysis of the reduction process of the iron ore. As a result, the reduction degree can be assessed from both thermogravimetric data and gas composition data, allowing for cross-validation and complementary information acquisition.

2. Hermetic Design with Pressure-Relief Buffering:

In the present disclosure, a highly hermetic reaction chamber is employed, and a gas convection buffer chamber is disposed above the exhaust gas outlet. Such configuration effectively mitigates disturbances to the reaction crucible caused by high-pressure, high-velocity, and high-temperature gas backflow, thereby achieving gas deceleration, pressure reduction, and cooling, and ensuring the accuracy of thermogravimetric analysis and exhaust gas analysis.

### 3. Real-Time Monitoring of Reaction Atmosphere:

An external gas analysis and monitoring device is utilized to monitor the real reaction atmosphere in the furnace chamber in real time, including changes in gas composition, concentration, and flow rate.

### 4. Accurate Assessment of Reduction Degree:

The reduction degree of the iron ore can be accurately converted and the contribution of different reducing gas reactions such as CO—CO2 and H2-H2O can be distinguished by the analysis of the composition of the exhaust gas.

5. Suppression of Interference from Non-Reducing Reaction:

In the present disclosure, the influence of non-reducing reactions, such as coke gasification or iron carburization resulting in Fe₃C formation, on weight measurement is eliminated. Accordingly, experimental accuracy is significantly improved.

### 6. Elimination of the Influence of Thermal Disturbance:

Interference with balance measurements caused by thermal convection or the crucible oscillation (pendulum effect) induced by furnace heating is effectively prevented by the exhaust gas analysis.

### 7. Data Processing Method:

The present disclosure comprises a set of data processing methods for extracting and calculating reduction degree of the iron ore and the contributions of various gas reactions from results of the exhaust gas analysis.

### 8. Applicability to Experiment in a Mixed Atmosphere:

Iron ore reduction experiments conducted in a mixed atmospheres containing multiple reducing gases can be accurately analyzed, thereby providing more flexible and comprehensive range of applications.

The present disclosure is characterized at least in that: (1) real-time and accurate monitoring of the reaction atmosphere and the reduction degree is enabled by the integration of analysis of the exhaust gas and the thermogravimetric analysis; (2) by a hermetic design and a pressure-relief buffer chamber mechanism, an impact of the high-temperature and high-pressure gases on the oscillation of the reaction crucible is effectively reduced, and measurement accuracy is improved, while hermeticity is maintained; and (3) applicability to scenarios involving simultaneous reactions with multiple reducing gases is provided, wherein the individual contributions of different reduction reactions to the overall reduction degree can be distinguished through analysis of the exhaust gas.

While the preferred embodiments of the present disclosure have been described above, it will be recognized and understood that various changes and modifications can be made, and the appended claims are intended to cover all such changes and modifications which may fall within the spirit and scope of the present disclosure.

1. A device for assessing reduction degree of iron ore in a mixed reducing atmosphere, comprising:
a hermetic heating device configured to heat a reaction sample;
an air intake device connected to the hermetic heating device, and configured to introduce an inert gas and/or a reducing gas into the hermetic heating device;
a gas analysis device configured to analyze a part of exhaust gas produced by the hermetic heating device;
a thermogravimetric analysis device configured to analyze a weight change of the hermetic heating device caused by reduction;
a gas convection buffer section disposed between the hermetic heating device and the thermogravimetric analysis device, and configured to mitigate disturbance caused by gas reflux on measurement of the thermogravimetric analysis device; and
a gas split device disposed between the hermetic heating device and the gas analysis device, and configured to split the exhaust gas produced by the hermetic heating device, thereby maintaining the part of exhaust gas introduced into the gas analysis device at a constant flow rate or a constant flow,
wherein a reduction degree of iron ore of the reaction sample in different reducing atmospheres is respectively assessed according to a measured value of the gas analysis device and a measured value of the thermogravimetric analysis device.

2. The device of claim 1, wherein the gas convection buffer section includes a gas convection buffer area and a constant pressure area, the constant pressure area is disposed near one side of the thermogravimetric analysis device, and the gas convection buffer area has a convection gas exhaust port configured to exhaust convection gas in the gas convection buffer area.

3. The device of claim 2, wherein a pressure in the constant pressure area is controlled to be 1 atmosphere.

4. The device of claim 2, wherein the hermetic heating device includes a reaction sample loading device configured to load the reaction sample, and the reaction sample is iron ore to be tested.

5. The device of claim 4, wherein the constant pressure area includes a hermetic component, a connector is disposed inside the hermetic component, one end of the connector is connected to the thermogravimetric analysis device, and the other end of the connector is connected to the reaction sample loading device.

6. A method for assessing reduction degree of iron ore in a mixed reducing atmosphere by using the device of claim 1, comprising steps of:
providing a reaction sample in a hermetic heating device to heat the reaction sample;
introducing an inert gas and/or a reducing gas into the hermetic heating device; and
introducing a part of exhaust gas produced by the hermetic heating device into a gas analysis device, and assessing reduction degree of iron ore of the reaction sample in different reducing atmospheres respectively according to a measured value of the gas analysis device and a measured value of the thermogravimetric analysis device.

7. The method of claim 6, wherein the reduction degree is obtained by assessing the reduction degree of the iron ore in different reducing atmospheres respectively by analyzing change in thermogravimetric weight loss to calculate the reduction degree of the iron ore using the thermogravimetric analysis device and by analyzing change in a composition of the exhaust gas using the gas analysis device.

8. The method of claim 7, wherein assessing the reduction degree of the iron ore by the change in thermogravimetric weight loss is performed by the following formula:
$$Reduction Degree ( RD ) = [ \frac{0.111 \times {W}_{1}}{0.43 \times {W}_{2}} + \frac{{M}_{1} - {M}_{t}}{{M}_{0} \times ( 0.43 \times {W}_{2} )} ] \times 100 \% ,$$
wherein RD % represents the reduction degree of the iron ore (%); M₀ represents a total mass of the iron ore; W₁ represents FeO content in iron ore (%); W₂ represents a total iron fraction (TFe) content of the iron ore (%); M₁ represents a total weight of the iron ore at the time t=0 of an experiment; and Mt represents a total weight of iron ore at the t^{th} second of an experiment.

9. The method of claim 7, wherein assessing the reduction degree of the iron ore in different reducing atmospheres respectively by utilizing gas change by analyzing the composition of the exhaust gas using the gas analysis device is performed by the following formula:
$$RD \% ( CO ) = \frac{{N}_{C O} + 2 {N}_{CO 2} - {N}_{introduced CO} - 2 {N}_{introduced CO 2}}{{M}_{0} \times [ ( 0.430 \times {W}_{2} ) - ( 0.111 \times {W}_{1} ) ] / 16} \times 100 \% ,$$
$$RD \% ( H 2 ) = \frac{{N}_{H 2} + 2 {N}_{H 2 O} - {N}_{introduced H 2} - 2 {N}_{introduced H 2 O}}{{M}_{0} \times [ ( 0.430 \times {W}_{2} ) - ( 0.111 \times {W}_{1} ) ] / 16} \times 100 \% ,$$
wherein RD % represents the reduction degree of the iron ore (%); M₀ represents a total mass of the iron ore; N_{CO} represents a molar amount of CO gas contained in the exhaust gas; N_{CO2} represents a molar amount of CO₂ gas contained in the exhaust gas; N_{introduced CO} represents a molar amount of CO gas contained in the introduced gas; N_{introduced cO2} represents a molar amount of CO₂ gas contained in the introduced gas; N_{H2} represents a molar amount of H₂ gas contained in the exhaust gas; N_{H2O} represents a molar amount of H₂O gas contained in the exhaust gas; N_{introduced H2} represents a molar amount of H₂ gas contained in the introduced gas; N_{introduced H2O} represents a molar amount of H₂O gas contained in the introduced gas; W₁ represents FeO content of the iron ore (%); and W₂ represents a total iron content (TFe) of the iron ore (%).

10. The method of claim 6, wherein an accuracy of the assessing of the reduction degree is verified by comparing the measured value of the gas analysis device with the measured value of the thermogravimetric analysis device.
